(19) United States
(12) Patent Application Publication
(10) Pub. No.: US 2018/0254098 A1
(43) Pub. Date: Sep. 6, 2018

(54) METHOD AND DATA PROCESSING UNIT FOR SELECTING A PROTOCOL FOR A MEDICAL IMAGING EXAMINATION

(71) Applicant: Siemens Healthcare GmbH, Erlangen (DE)

(72) Inventors: Thomas ALLMENDINGER, Forchheim (DE); Matthias BAER, Erlangen (DE); Ulrike HABERLAND, Erlangen (DE); Christiane KOCH, Eggolsheim (DE); Sebastian SCHMIDT, Weisendorf (DE); Ute FEUERLEIN, Erlangen (DE); Rainer RAUPACH, Heroldsbach (DE)

(73) Assignee: Siemens Healthcare GmbH, Erlangen (DE)

(21) Appl. No.: 15/905,909

(22) Filed: Feb. 27, 2018

(30) Foreign Application Priority Data
Mar. 1, 2017 (DE) 102017203315.0

(51) Int. Cl. G16H 30/20; G06N 99/00

(52) U.S. Cl. CPC G16H 30/20; G06N 99/005

(57) ABSTRACT

A method is for selecting a protocol for a medical imaging examination. In an embodiment, the method includes providing a plurality of protocols; providing a classification system for medical imaging examinations having a plurality of hierarchically ordered categories; determining a node from the quantity of nodes belonging to the set of nodes by which the medical imaging examination can be identified, and to which one protocol respectively is assigned whose category relative to the categories of the other nodes of this quantity is lowest; and selecting the protocol, assigned to the determined node, for the medical imaging examination.

### PRIORITY STATEMENT

The present application hereby claims priority under 35 U.S.C. § 119 to German patent application number DE 102017203315.0 filed Mar. 1, 2017, the entire contents of which are hereby incorporated herein by reference.

### FIELD

At least one embodiment of the invention generally relates to a method for selecting a protocol for a medical imaging examination. At least one embodiment of the invention also generally relates to a data processing unit, to a medical imaging device, to a computer program and to a computer-readable medium.

### BACKGROUND

The depth of specialization of examination protocols in imaging methods can vary greatly depending on the clinical issue. Therefore there are, for example, very general protocols, which are also called routine protocols, which can be applied to a very large number of possible issues. On the other hand, there are also very specific protocols for dedicated issues. The type and degree of specialization can be very different from user to user, moreover.

Different examination protocols are conventionally stored as lists, in particular linearly. Each examination protocol is identified by a unique name. In principle the clinical purpose can also be encoded in rudimentary form by way of the name. In particular in the case of ambiguities, resulting due to different depths of specialization, the choice of protocol appropriate to the issue can be made, for example manually, based on additional rules.

Rules, with which the existing protocols are assigned to the issues for each user specifically, can be compiled in a catalogue of rules, also called a protocol cookbook. A catalogue of rules of this kind is conventionally stored on the control console of the medical imaging device as a printed and/or an electronic document. An examination protocol is assigned to an examination request as a function of the examination request and with the aid thereof by the user of the medical imaging device.

The rules are often device-dependent even within the same imaging modality and have to be individually created for each device and/or be provided on the device. This procedure is relatively laborious and prone to errors, in particular because it is not automatically guaranteed that the rules will also be correctly implemented by the user. The theoretical intermediate step with additional rules for protocol selection can, in principle, be avoided in that for each conceivable clinical issue, protocols are created with a high level of detail, in particular with a high degree of redundancy, which can then be selected directly by way of its name for each issue.

### SUMMARY

At least one embodiment of the invention enables a simplified selection of examination protocols for a medical imaging examination.

Further advantageous embodiments of the invention are considered in the claims.

At least one embodiment of the invention relates to a method for selecting a protocol for a medical imaging examination, the method comprising:
providing a plurality of protocols,
providing a classification system for medical imaging examinations, having a plurality of hierarchically ordered categories,
   wherein each category has at least one node, which is assigned to a node of a next higher category and/or to which at least one node of a next lower category is assigned,
   wherein the medical imaging examination can be identified by a set of nodes, which has at most one node from each category of the plurality of categories,
   wherein the classification system has a plurality of nodes, to which one protocol respectively of the plurality of protocols is assigned,
determining a node from the quantity of those nodes, which belong to the set of nodes by which the medical imaging examination can be identified, and to which one protocol respectively is assigned whose category relative to the categories of the other nodes of this quantity is lowest,
selecting the protocol, which is assigned to the determined node, for the medical imaging examination.

At least one embodiment of the invention also relates to a data processing unit for selecting a protocol for a medical imaging examination, having:
a protocol providing unit for providing a plurality of protocols,
a classification system providing unit for providing a classification system for medical imaging examinations, having a plurality of hierarchically ordered categories,
wherein each category has at least one node, which is assigned to a node of a next higher category and/or to which at least one node of a next lower category is assigned,
wherein the medical imaging examination can be identified by a set of nodes, which has at most one node from each category of the plurality of categories,
wherein the classification system has a plurality of nodes, to which one protocol respectively of the plurality of protocols is assigned,
a node determining unit for determining a node from the quantity of nodes, which belong to the set of nodes by which the medical imaging examination can be identified, and to which one protocol respectively is assigned whose category relative to the categories of the other nodes of this quantity is lowest,
a protocol selecting unit for selecting the protocol, which is assigned to the determined node, for the medical imaging examination.

At least one embodiment of the invention also relates to a medical imaging device, having a data processing unit as claimed in one of the embodiments, which are disclosed in this application.

At least one embodiment of the invention also relates to a computer program, which can be loaded into a storage device of a data processing system, having program segments in order to carry out all steps of a method of one of the embodiments, which are disclosed in this application, when the computer program is run by the data processing system.

At least one embodiment of the invention also relates to a computer-readable medium on which program segments, which can be read and run by a data processing system, are stored in order to carry out all steps of a method of one of the embodiments, which are disclosed in this application, when the program segments are run by the data processing system.

A further embodiment of the invention provides that the classification system has more than three categories or less than three categories. One embodiment of the invention provides that each node of the respective category is assigned to a node of the next higher category for each category with the exception of the highest category.

According to one embodiment of the invention, the medical imaging device has an acquisition unit, which is designed for acquisition of the acquisition data. In particular, the acquisition unit can have a source of radiation and a radiation detector. One embodiment of the invention provides that the source of radiation is designed for emission and/or excitation of radiation, in particular electromagnetic radiation, and/or that the radiation detector is designed for detection of the radiation, in particular the electromagnetic radiation. The radiation can pass for example from the source of radiation to a region to be depicted and/or after interaction with the region to be depicted to the radiation detector. The radiation is modified during interaction with the region to be depicted and therewith becomes the carrier of information, which relates to the image to be depicted. This information is acquired in the form of acquisition data during interaction of the radiation with the detector.

In an embodiment, one component of the data processing unit in one of the embodiments, which is disclosed in this application, which is designed to carry out a given step of a method as claimed in one of the embodiments, which are disclosed in this application, can be implemented in the form of hardware, which is configured for carrying out the given step and/or which is configured for carrying out a computer-readable instruction in such a way that the hardware can be configured by way of the computer-readable instruction to carry out the given step. In particular, the system can have a storage area, for example in the form of a computer-readable medium, in which computer-readable instructions, for example in the form of a computer program, are stored.

The computer program product according to one of the embodiments, which is disclosed in this application, and/or the computer program according to one of the embodiments, which is disclosed in this application, for example can be stored on the computer-readable medium. The computer-readable medium can be for example a memory stick, a hard disk or another data carrier, which can in particular be detachably connected to the data processing system or be permanently integrated in the data processing system. The computer-readable medium can for example form a region of the storage system of the data processing system.

### BRIEF DESCRIPTION OF THE DRAWINGS

Selected embodiments of the invention will be illustrated below with reference to the accompanying figures. The illustration in the figures is schematic, highly simplified and not necessarily to scale.

In the drawings:

FIG. 1 shows a schematic illustration of an example classification system,

FIG. 2 shows a schematic illustration of an assignment of examination protocols to nodes of a further example classification system,

FIG. 3 shows a schematic illustration of a selection of an examination protocol for a medical imaging examination according to one embodiment of the invention,

FIG. 4 shows a flowchart for a method for the selection of an examination protocol for a medical imaging examination according to a further embodiment of the invention,

FIG. 5 shows a schematic illustration of a data processing unit for selection of an examination protocol for a medical imaging examination according to a further embodiment of the invention,

FIG. 6 shows a flowchart for a method for selection of an examination protocol for a medical imaging examination according to a further embodiment of the invention,

FIG. 7 shows a schematic illustration of a data processing unit for selection of an examination protocol for a medical imaging examination according to a further embodiment of the invention and

FIG. 8 shows a schematic illustration of a medical imaging device according to a further embodiment of the invention.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

At least one embodiment of the invention relates to a method for selecting a protocol for a medical imaging examination, the method comprising:
providing a plurality of protocols,
providing a classification system for medical imaging examinations, having a plurality of hierarchically ordered categories,
   wherein each category has at least one node, which is assigned to a node of a next higher category and/or to which at least one node of a next lower category is assigned,
   wherein the medical imaging examination can be identified by a set of nodes, which has at most one node from each category of the plurality of categories,
   wherein the classification system has a plurality of nodes, to which one protocol respectively of the plurality of protocols is assigned,
determining a node from the quantity of those nodes, which belong to the set of nodes by which the medical imaging examination can be identified, and to which one protocol respectively is assigned whose category relative to the categories of the other nodes of this quantity is lowest,
selecting the protocol, which is assigned to the determined node, for the medical imaging examination.

In an embodiment, the medical imaging examination can be a medical computerized tomography imaging examination. In particular, the medical imaging examinations can be medical computerized tomography imaging examinations.

In an embodiment, the classification system can have at least three categories and/or exactly three categories.

In an embodiment, the classification system can have one or more categories, which are chosen from the group of categories, which comprises a first category, which relates to a region of the body to be examined, a second category, which relates to an anatomical focus of the medical imaging examination, and a third category, which relates to an issue of the medical imaging examination.

In an embodiment, the method can also comprise the following steps:
providing an examination request, which relates to the medical imaging examination,
determining the set of nodes by which the medical imaging examination can be identified, based on the examination request.

In an embodiment, the method can also comprise the following steps:
providing a set of training data records, wherein each training data record of the set of training data records has an examination request for medical imaging,
determining the classification system based on the set of training data records and a machine learning algorithm.

In an embodiment, each training data record of the set of training data records can have a protocol assigned to the examination request.

In an embodiment, the protocols of the plurality of protocols can be assigned to the nodes of the plurality of nodes based on the set of training data records and a machine learning algorithm.

In an embodiment, the set of training data records has examination requests and/or protocols of at least two different medical imaging devices, by which the medical imaging examination can be carried out in each case.

At least one embodiment of the invention also relates to a data processing unit for selecting a protocol for a medical imaging examination, having:
a protocol providing unit for providing a plurality of protocols,
a classification system providing unit for providing a classification system for medical imaging examinations, having a plurality of hierarchically ordered categories,
wherein each category has at least one node, which is assigned to a node of a next higher category and/or to which at least one node of a next lower category is assigned,
wherein the medical imaging examination can be identified by a set of nodes, which has at most one node from each category of the plurality of categories,
wherein the classification system has a plurality of nodes, to which one protocol respectively of the plurality of protocols is assigned,
a node determining unit for determining a node from the quantity of nodes, which belong to the set of nodes by which the medical imaging examination can be identified, and to which one protocol respectively is assigned whose category relative to the categories of the other nodes of this quantity is lowest,
a protocol selecting unit for selecting the protocol, which is assigned to the determined node, for the medical imaging examination.

In an embodiment, the data processing unit can also have the following components:
an examination request-providing unit for providing an examination request, which relates to the medical imaging examination,
a node set determining unit for determining the set of nodes by which the medical imaging examination can be identified, based on the examination request.

In an embodiment, the data processing unit can also have the following components:
a training data record providing unit for providing a set of training data records, wherein each training data record of the set of training data records has an examination request for medical imaging,
a classification system determining unit for determining the classification system based on the set of training data records and a machine learning algorithm.

In an embodiment, the data processing unit can be designed to carry out a method of one of the embodiments which are disclosed in this application.

At least one embodiment of the invention also relates to a medical imaging device, having a data processing unit as claimed in one of the embodiments, which are disclosed in this application.

In an embodiment, the medical imaging device can be selected from the imaging modalities group including an X-ray device, a C-arm X-ray device, a computerized tomography device, a molecular imaging device, a single photon emission computerized tomography device, a positron emission tomography device, a magnetic resonance tomography device and combinations thereof.

At least one embodiment of the invention also relates to a computer program, which can be loaded into a storage device of a data processing system, having program segments in order to carry out all steps of a method of one of the embodiments, which are disclosed in this application, when the computer program is run by the data processing system.

At least one embodiment of the invention also relates to a computer-readable medium on which program segments, which can be read and run by a data processing system, are stored in order to carry out all steps of a method of one of the embodiments, which are disclosed in this application, when the program segments are run by the data processing system.

In an embodiment, a hierarchical classification system can be defined, with which clinical issues, which are possible for a medical imaging examination, can be sufficiently precisely described. The degree of specialization of the hierarchically ordered categories increases from top to bottom or remains the same.

The degree of specialization of the hierarchically ordered categories increases in particular if the quantity of nodes in the next lower category, which can be consulted for identification of a medical imaging examination, is restricted due to the confinement to a node in one category. Of course, it is not impossible for the quantity of nodes in the next lower category, which can be consulted for identification of a medical imaging examination, to not be restricted due to a confinement to a node in one category.

A further embodiment of the invention provides that the classification system has more than three categories or less than three categories. One embodiment of the invention provides that each node of the respective category is assigned to a node of the next higher category for each category with the exception of the highest category.

Examination protocols can be suitably assigned to specific nodes of the classification system.

In an embodiment, it is not necessary for one examination protocol respectively to be assigned to all nodes of the classification system. If, for example, no examination protocol is assigned to a first node, an examination protocol, which is assigned to a second node, can therefore be used for a medical imaging examination, which can be identified using this first node, with the second node belonging to a higher category and with the first node being directly or indirectly assigned to the second node.

In an embodiment, the classification system can be provided by defining the hierarchically ordered categories, in particular manually and/or based on a machine learning algorithm.

The inventive solution of at least one embodiment also enables automation of the selection of examination protocols based on machine learning. With knowledge of the protocols that exist in a hospital and their use in the context of an examination request via the HIS/RIS (Hospital Information System/Radiology Information System), a classification system can be determined for example based on corresponding training data records and a machine learning algorithm, here for example Recursive Partitioning Tree Learning. Furthermore, the examination protocols can be assigned to the nodes of the classification system by way of the machine learning algorithm. It is thereby possible to automatically select examination protocols on the basis of the examination request.

In the context of this application, a machine learning algorithm is in particular taken to mean an algorithm, which is designed for machine learning. A machine learning algorithm can be implemented for example with the aid of decision trees, mathematical functions and/or general programming languages. The machine learning algorithm can be designed for example for supervised learning and/or for unsupervised learning. The machine learning algorithm can be designed for example for deep learning and/or for reinforcement learning and/or for Marginal Space Learning. In particular with supervised learning, a function category can be used, which is based, for example, on decision trees, a Random Forest, a logistical regression, a Support Vector Machine, an artificial neural network, a kernel method, Bayes classifiers or similar or combinations thereof.

Possible implementations of the machine learning algorithm can use, for example, artificial intelligence. Alternatively or in addition to the first machine learning algorithm and/or the second machine learning algorithm, one or more rule-based algorithms can be used. Calculations, in particular when determining the classification system based on the set of training data records and a machine learning algorithm, can be made, for example, via a processor system. The processor system can have, for example, one or more graphics processors.

In an embodiment, data, which relates for example to a medical image, a protocol or a classification system, can be provided by loading the data, for example from a region of a storage system, and/or be generated, for example generated via a medical imaging device. In particular, one step or a plurality of steps or all steps of an embodiment of the inventive method can be carried out automatically and/or via a component of a data processing unit, with the component being formed for example by a processor system. In particular, the medical imaging examination can be an examination via a medical imaging device and/or be carried out via a medical imaging device.

The medical imaging device can be chosen for example from the imaging modalities group, which consists of an X-ray device, a C-arm X-ray device, a computerized tomography device (CT device), a molecular imaging device (MI device), a single photon emission computerized tomography device (SPECT device), a positron emission tomography device (PET device), a magnetic resonance tomography device (MR device) and combinations thereof, in particular a PET-CT device and a PET-MR device. The medical imaging device can also have a combination of an imaging modality, which is selected for example from the imaging modalities group, and an irradiation modality. The irradiation modality can have for example an irradiation unit for therapeutic irradiation. Without restricting the general inventive idea, a computerized tomography device is cited by way of example for a medical imaging device in some of the embodiments.

According to one embodiment of the invention, the medical imaging device has an acquisition unit, which is designed for acquisition of the acquisition data. In particular, the acquisition unit can have a source of radiation and a radiation detector. One embodiment of the invention provides that the source of radiation is designed for emission and/or excitation of radiation, in particular electromagnetic radiation, and/or that the radiation detector is designed for detection of the radiation, in particular the electromagnetic radiation. The radiation can pass for example from the source of radiation to a region to be depicted and/or after interaction with the region to be depicted to the radiation detector. The radiation is modified during interaction with the region to be depicted and therewith becomes the carrier of information, which relates to the image to be depicted. This information is acquired in the form of acquisition data during interaction of the radiation with the detector.

In an embodiment with a computerized tomography device and with a C-arm X-ray device, the acquisition data can be projection data, the acquisition unit a projection data acquisition unit, the source of radiation an X-ray source, the radiation detector an X-ray detector. The X-ray detector can in particular be a quantum-counting and/or energy-resolving X-ray detector. In particular with a magnetic resonance tomography device, the acquisition data can be a magnetic resonance data set, the acquisition unit a magnetic resonance data acquisition unit, the source of radiation a first radio frequency antenna unit, the radiation detector the first radio frequency antenna unit and/or a second radio frequency antenna unit.

The data processing unit and/or one or more component(s) of the data processing unit can be formed by a data processing system. The data processing system can have, for example, one or more components in the form of hardware and/or one or more components in the form of software. The data processing system can be formed for example at least partially by a cloud computing system. The data processing system can be and/or have for example a cloud computing system, a computer network, a computer, a tablet, a Smartphone or the like or combinations thereof.

The hardware can cooperate for example with software and/or be configured by way of software. The software can be run for example by way of the hardware. The hardware can be for example a storage system, an FPGA system (field-programmable gate array), an ASIC system (application-specific integrated circuit), a microcontroller system, a processor system and combinations thereof. The processor system can have for example a microprocessor and/or a plurality of cooperating microprocessors.

In an embodiment, one component of the data processing unit in one of the embodiments, which is disclosed in this application, which is designed to carry out a given step of a method as claimed in one of the embodiments, which are disclosed in this application, can be implemented in the form of hardware, which is configured for carrying out the given step and/or which is configured for carrying out a computer-readable instruction in such a way that the hardware can be configured by way of the computer-readable instruction to carry out the given step. In particular, the system can have a storage area, for example in the form of a computer-readable medium, in which computer-readable instructions, for example in the form of a computer program, are stored.

Data can be transferred between components of the data processing system for example via a suitable data transfer interface in each case. The data transfer interface for data transfer to and/or from a component of the data processing system can be implemented at least partially in the form of software and/or at least partially in the form of hardware. The data transfer interface can be designed for example for storing data in and/or for loading data from a region of the storage system, it being possible to access one or more components of the data processing system on this region of the storage system.

The computer program can be loaded into the storage system of the data processing system and be run by the processor system of the data processing system.

The data processing system can be designed for example by way of the computer program in such a way that the data processing system can carry out the steps of a method according to one of the embodiments, which are disclosed in this application, when the computer program is run by the data processing system.

The computer program product according to one of the embodiments, which is disclosed in this application, and/or the computer program according to one of the embodiments, which is disclosed in this application, for example can be stored on the computer-readable medium. The computer-readable medium can be for example a memory stick, a hard disk or another data carrier, which can in particular be detachably connected to the data processing system or be permanently integrated in the data processing system. The computer-readable medium can for example form a region of the storage system of the data processing system.

According to one embodiment of the invention, a protocol is assigned to at least one node of the set of nodes by which the medical imaging examination can be identified. In the context of this application the terms protocol and examination protocol are used synonymously.

In the context of at least one embodiment of the invention, features, which are described in respect of different embodiments of the invention and/or different categories of claims (method, use, device, system, arrangement, etc.), are combined to form further embodiments of the invention. For example, an embodiment, which relates to a device, can also be developed with features that are described or claimed in conjunction with a method. Functional features of a method can be implemented by appropriately designed concrete components. In addition to the embodiments of the invention expressly described in this application, a wide variety of further embodiments of the invention is conceivable, at which a person skilled in the art can arrive without departing from the scope of the invention insofar as it is specified by the claims.

Use of the indefinite article “a” or “an” does not preclude the relevant feature from also being present multiple times. Use of the expression “to have” does not preclude the terms linked by way of the expression “to have” from being identical. For example, the medical imaging device has the medical imaging device. Use of the expression “unit” does not preclude the article, to which the expression “unit” refers, from having a plurality of components which are spatially separated from each other.

In the context of the present application, the expression “based on” can in particular be taken understood within the meaning of the expression “using”. In particular wording, which is generated as a result of a first feature based on a second feature (alternative: ascertained, determined, etc.), does not preclude the first feature from being generated on the basis of a third feature (alternative: ascertained, determined, etc.).

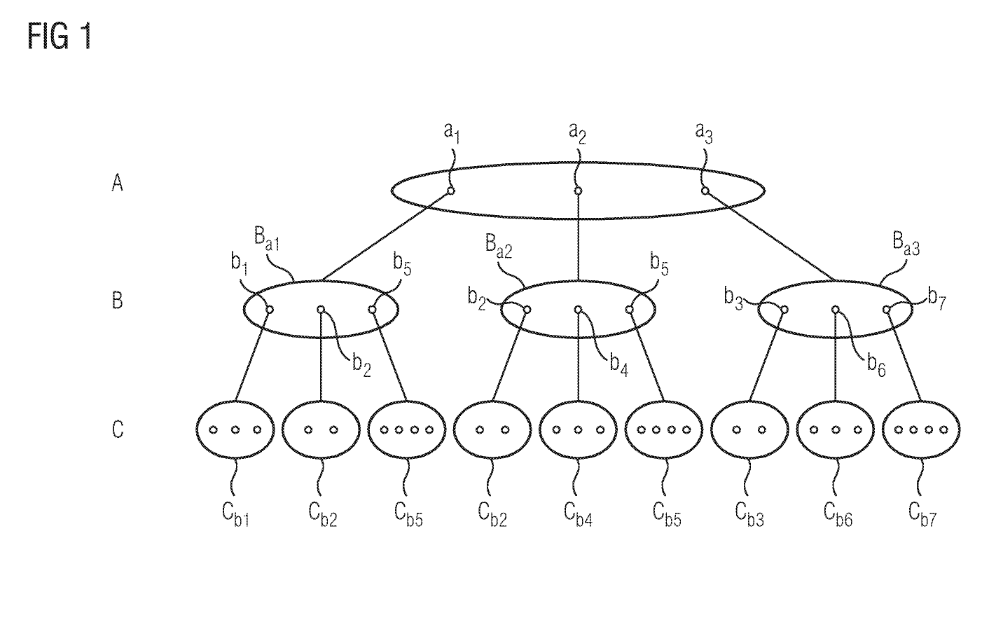

FIG. 1 shows a schematic illustration of an example classification system. The classification system comprises a categories triple, which comprises a category A, which for example relates to a region of the body to be examined, a category B, which relates for example to an anatomical focus of the medical imaging examination, and a category C, which relates to an issue, which for example relates to a clinical indication, of the medical imaging examination.

Category A has the nodes aᵢ, i=1, . . . , 3. Category B has the nodes bⱼ, j=1, . . . , 7. For each aᵢϵA there is a subset Bₐᵢ ⊆B in which the nodes appropriate to ai are located. For each bⱼϵB there is a subset C_{bj}⊆C in which the nodes appropriate to bⱼ are located, etc. Of course it is not impossible for there to be an aᵢ, for which Bₐᵢ=B applies.

The categories can in particular have the quantities of nodes given below.

A={head, neck, shoulder, thorax, abdomen, . . . }
B={brain, sinus, eye socket, carotid, larynx, shoulder joint, . . . }
C={mass, seizure, headache symptoms, fracture, . . . }

With confinement to the node a₁=head in category A, for example the quantity of nodes in category B, which can be consulted for identification of a medical imaging examination, can be restricted to the subset Bₐ₁={brain, sinus, eye socket}⊆B.

With confinement to the node b₁=brain in category B, for example the quantity of nodes in category C, which can be consulted for identification of a medical imaging examination, can be restricted to the subset C_{b1}={mass, seizure, headache symptoms}⊆C.

In particular, a maximally specialized medical imaging examination can be identified in this way by exactly one node respectively from each corresponding category. For example, a medical imaging examination can be identified by the node triple (a, b, c)=(head, brain, headache symptoms), with the conditions bϵBₐ and cϵC_{b} being met.

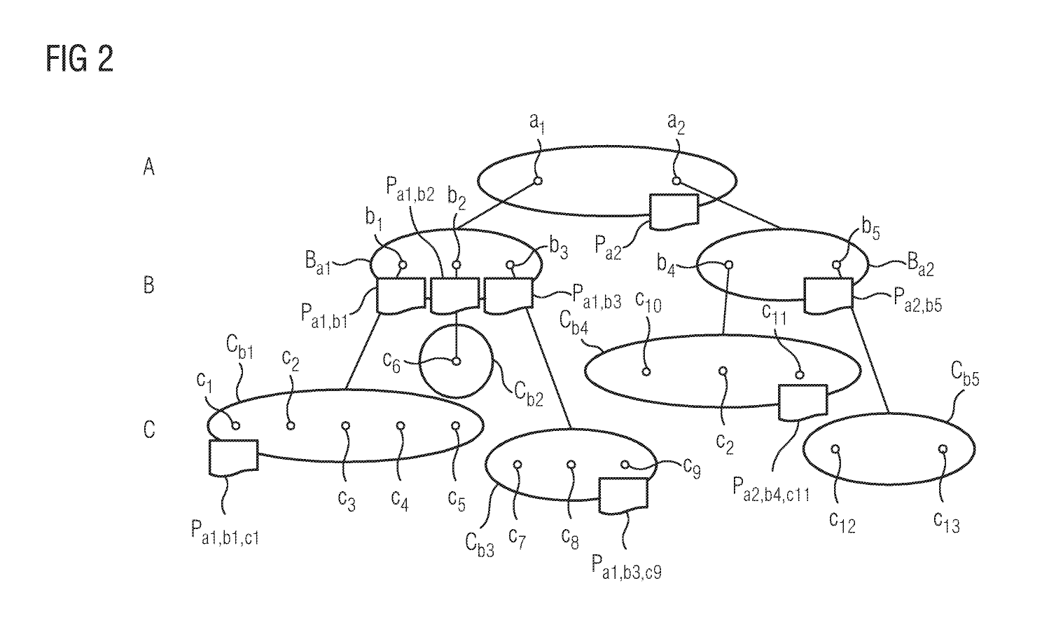

FIG. 2 shows a schematic illustration of an assignment of examination protocols to nodes of a further example classification system.

For example, one possible embodiment respectively of a medical imaging examination can be assigned as follows to each node.

a₁=head, a₂=abdomen, b₁=brain, b₂=sinus, b₃=temporal bone, b₄=liver, b₅=pancreas, c₁=stroke, c₂=metastasis, c₃=mass, c₄=headache symptoms, c₅=seizure, c₆=sinusitis, c₇=hearing loss, c₈=inflammation, c₉=cochlea implant, c₁₀=hypervascular tumor, c₁₁=hemangioma, c₁₂=pancreatitis, c₁₃=pancreas tumor.

The following protocols for example were assigned, which are known to a person skilled in the art in particular by the name stated in each case.

Pₐ₂=“Abdomen Routine (2-phasic)”, P_{a1, b1}=“Neuro Routine”, P_{a1, b2}=“Sinus”, P_{a1, b3}=“Temporal Bones”, P_{a2, b5}=“Pancreas (2-phasic)”, P_{a1, b1, c1}=“Brain Perfusion”, P_{a1, b3, c9}=“Inner Ear (UltraHR)”, P_{a2, b4, c1}=“Abdomen Routine (3-phasic)”.

In particular, it is not necessary for an examination protocol to be assigned to each node in the lowest category, which is used for identification of a maximally specialized medical imaging examination. For example, there are nodes in higher categories, to which one examination protocol respectively is assigned and to which nodes in lower categories are assigned to which no examination protocol is assigned.

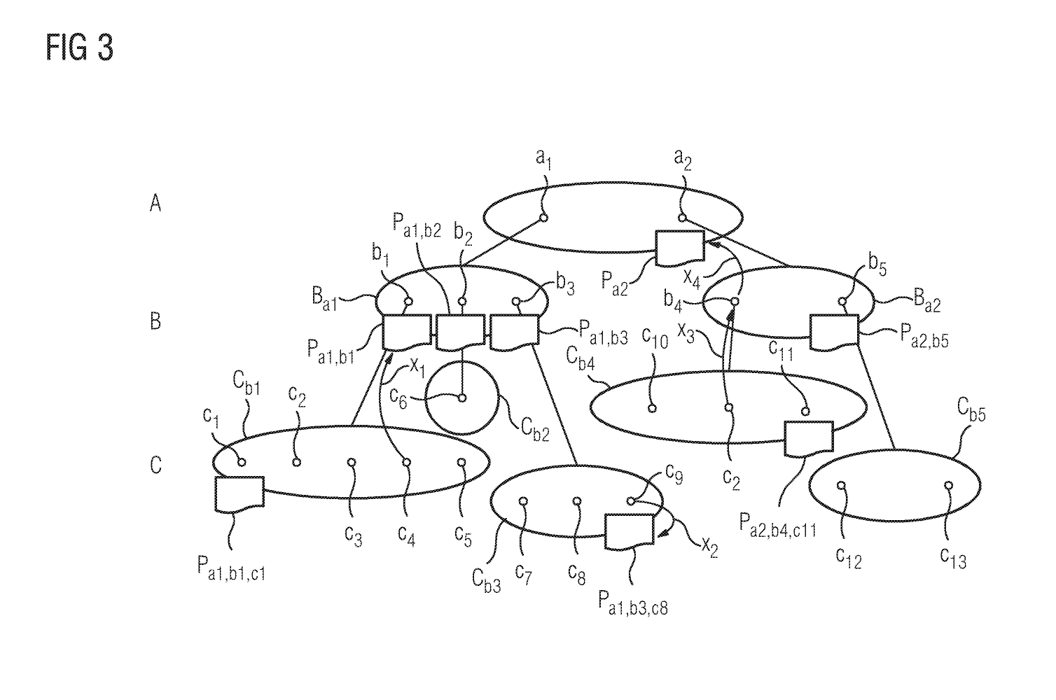

FIG. 3 shows a schematic illustration of a selection of an examination protocol for a medical imaging examination according to one embodiment of the invention. A protocol for a specific examination (a, b, c) is designated P_{a,b,c}. A protocol, which can be used unspecifically for all examinations (a, b, *) with any cϵC_{b}, is designated P_{a,b,*}, etc.

In addition, a protocol in the same category can be used for a plurality of nodes, in particular without having to be defined several times. A protocol that is used for two indications, c₁ϵC and c₂ϵC, is designated for example P_{a, b, c1|c2}.

The process is accordingly as follows for selecting a protocol for a specific examination (a, b, c).
When P_{a,b,c} is defined, select P_{a,b,c},
otherwise, when P_{a,b,*} is defined, select P_{a,b,*},
otherwise, when P_{a,*,*} is defined, select P_{a,*},

FIG. 3 shows the execution of these selection steps using by way of example the filled circles, which represent examinations, and the bent arrows x₁, x₂, x₃, and x₄, which indicate the examination protocols, which should be selected for the medical imaging examination accordingly. In other words, when a protocol is assigned to the node in the lowest category, which is consulted for identification of the medical imaging examination, this protocol is used, otherwise it is checked whether a protocol is assigned to the node in the next higher category. If a protocol is assigned to the node in the next higher category, then this protocol is used. If no protocol is assigned to the node in the next higher category, it is checked whether a protocol is assigned in the next but one higher category, etc. For example, node c₂=metastasis is directly assigned to node b₄=liver and therewith indirectly to node a₂=abdomen.

At least one embodiment of the inventive solution enables in particular a reduction in the number of examination protocols to be defined with simultaneous clear allocation of protocols to specific examinations. Furthermore, at least one embodiment of the inventive solution enables more structured information to be provided about the intended application of examination protocols and a possibility for their transfer together with the actual parameters of the examination protocol to application-specific use of the protocol on other imaging systems. Furthermore, at least one embodiment of the inventive solution enables extensive automation of protocol selection and a reduction in the number of protocols stored overall.

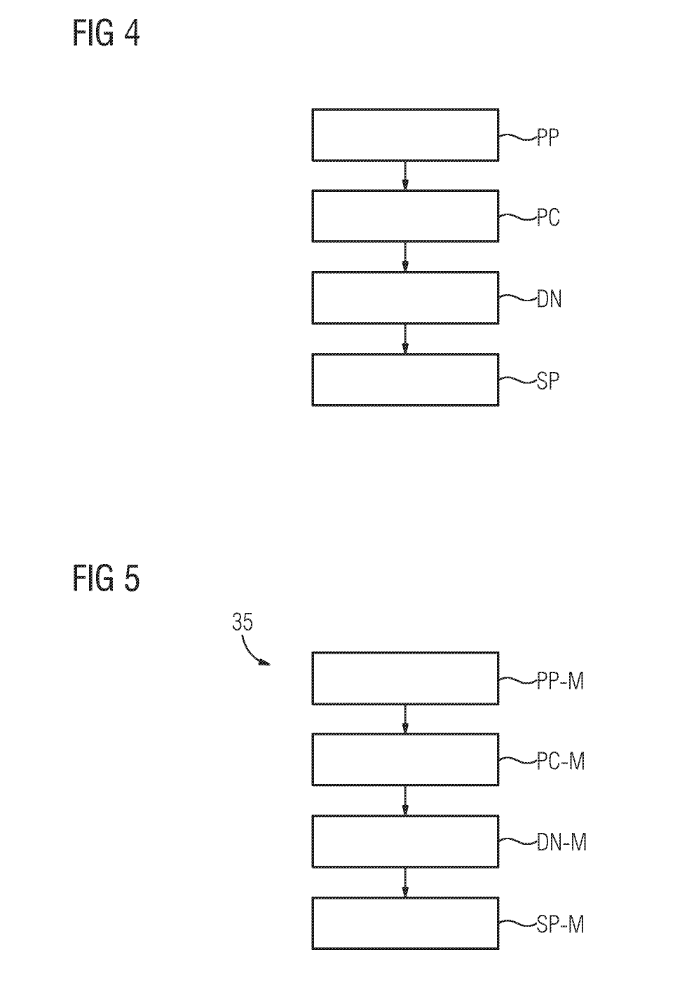

FIG. 4 shows a flowchart for a method for the selection of an examination protocol for a medical imaging examination according to a further embodiment of the invention, wherein the method comprises the following steps:
providing PP a plurality of protocols,
providing PC a classification system for medical imaging examinations having a plurality of hierarchically ordered categories,
wherein each category has at least one node, which is assigned to a node of a next higher category and/or to which at least one node of a next lower category is assigned,
wherein the medical imaging examination can be identified by a set of nodes, which at most has one node from each category of the plurality of categories,
wherein the classification system has a plurality of nodes, to which one protocol respectively of the plurality of protocols is assigned,
determining DN a node from the quantity of those nodes, which belong to the set of nodes by which the medical imaging examination can be identified, and to which one protocol respectively is assigned whose category relative to the categories of the other nodes of this quantity is lowest,
selecting SP the protocol, which is assigned to the determined node, for the medical imaging examination.

FIG. 5 shows a schematic illustration of a data processing unit 35 for selecting an examination protocol for a medical imaging examination according to a further embodiment of the invention, having:
a protocol providing unit PP-M designed for providing PP a plurality of protocols,
a classification system providing unit PC-M designed for providing PC a classification system for medical imaging examinations having a plurality of hierarchically ordered categories,
wherein each category has at least one node, which is assigned to a node of a next higher category and/or to which at least one node of a next lower category is assigned,
wherein the medical imaging examination can be identified by a set of nodes, which has at most one node from each category of the plurality of categories,
wherein the classification system has a plurality of nodes, to which one protocol respectively of the plurality of protocols is assigned,
a node determining unit DN-M designed for determining DN a node from the quantity of those nodes, which belong to the set of nodes by which the medical imaging examination can be identified, and to which one protocol respectively is assigned whose category relative to the categories of the other nodes of this quantity is lowest,
a protocol selecting unit SP-M designed for selecting SP the protocol, which is assigned to the determined node, for the medical imaging examination.

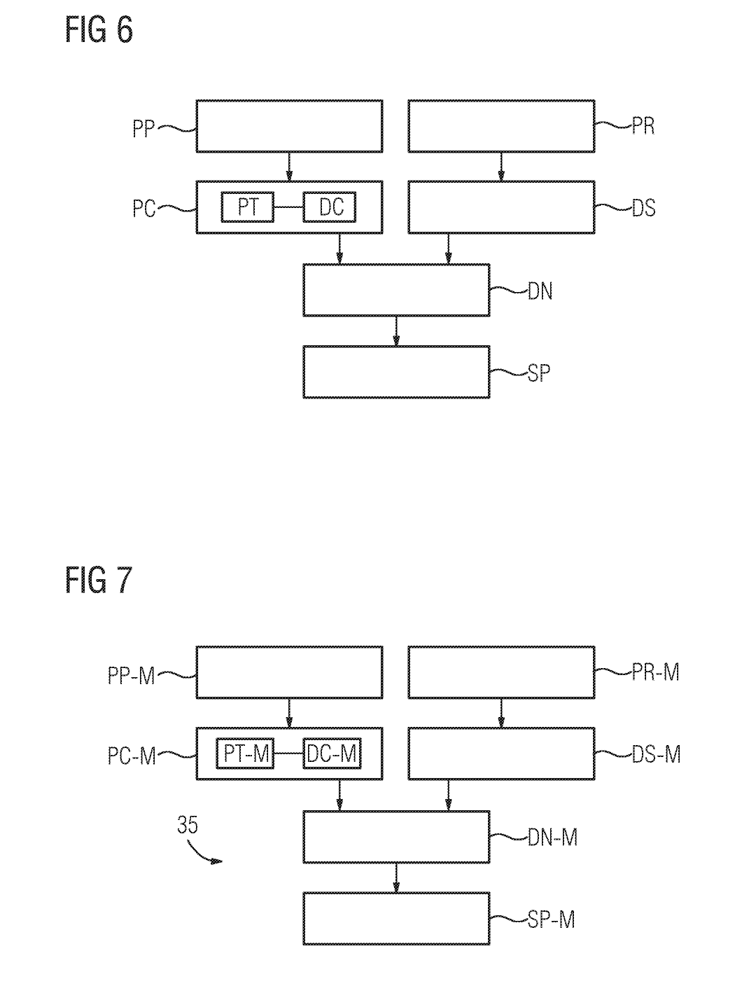

FIG. 6 shows a flowchart for a method for selecting an examination protocol for a medical imaging examination according to a further embodiment of the invention, wherein the method also comprises the following steps:
providing PR an examination request, which relates to the medical imaging examination,
determining DS the set of nodes by which the medical imaging examination can be identified based on the examination request,
providing PT a set of training data records, wherein each training data record of the set of training data records has an examination request for medical imaging,
determining DC the classification system based on the set of training data records and a machine learning algorithm.

FIG. 7 shows a schematic illustration of a data processing unit 35 for selecting an examination protocol for a medical imaging examination according to a further embodiment of the invention, also having:
an examination request-providing unit PR-M designed for providing PR an examination request, which relates to the medical imaging examination,
a node set determining unit DS-M designed for determining DS of the set of nodes by which the medical imaging examination can be identified based on the examination request,
a training data record providing unit PT-M designed for providing PT a set of training data records, wherein each training data record of the set of training data records has an examination request for medical imaging,
a classification system determining unit DC-M designed for determining DC the classification system based on the set of training data records and a machine learning algorithm.

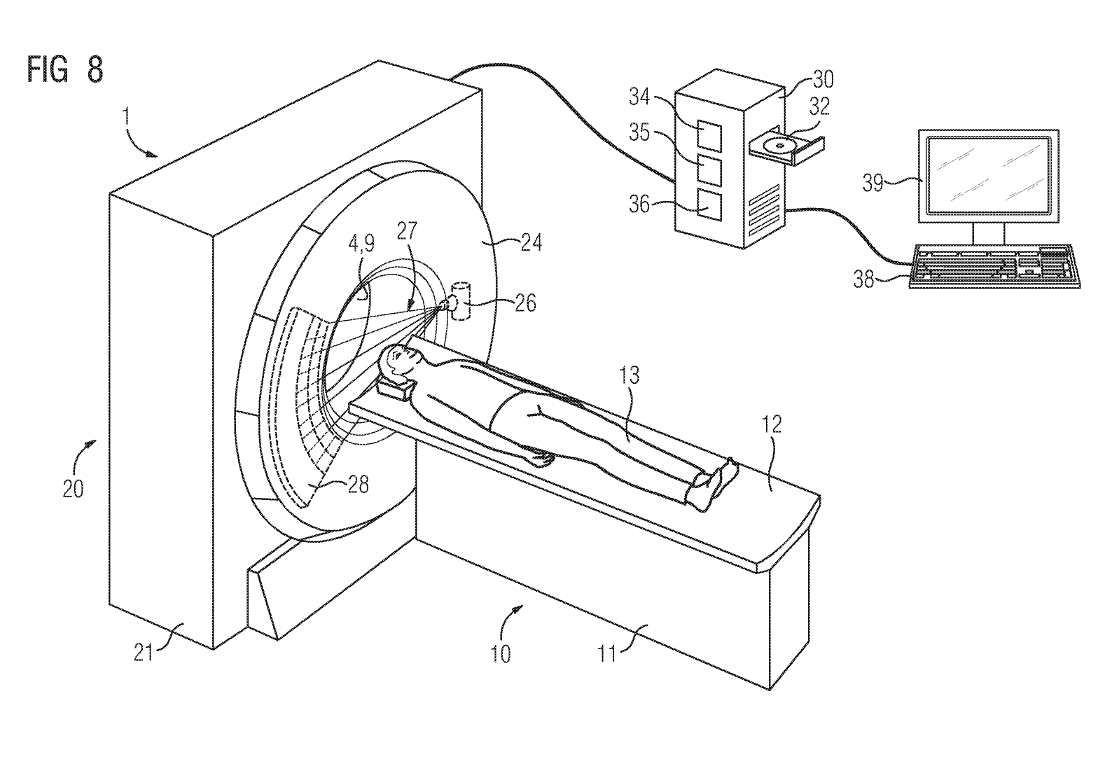

FIG. 8 shows a schematic illustration of a medical imaging device 1 according to a further embodiment of the invention. Without restricting the general inventive idea, a computerized tomography device is shown by way of example for the medical imaging device 1. The medical imaging device 1 has the gantry 20, the tunnel-like opening 9, the patient-supporting device 10 and the controller 30. The gantry 20 has the stationary support frame 21 and the rotor 24.

The patient 13 can be introduced into the tunnel-like opening 9. The acquisition region 4 is located in the tunnel-like opening 9. A region to be depicted of the patient 13 can be positioned in the acquisition region 4 in such a way that the radiation 27 from the source of radiation 26 can pass to the region to be depicted and after interaction with the region to be depicted can pass to the radiation detector 28.

The patient-positioning device 10 has the positioning base 11 and the positioning board 12 for positioning the patient 13. The positioning board 12 is arranged on the positioning base 11 so it can be moved relative to the positioning base 11 in such a way that the positioning board 12 can be introduced in a longitudinal direction of the positioning board 12, in particular along the system axis AR, into the acquisition region 4.

The medical imaging device 1 is designed for the acquisition of acquisition data based on electromagnetic radiation 27. The medical imaging device 1 has an acquisition unit. The acquisition unit is a projection data acquisition unit having the source of radiation 26, for example an X-ray source, and the detector 28, for example an X-ray detector, in particular an energy-resolving X-ray detector.

The source of radiation 26 is arranged on the rotor 24 and designed for the emission of radiation 27, for example X-ray radiation, with radiation quanta 27. The detector 28 is arranged on the rotor 24 and designed for detection of the radiation quanta 27. The radiation quanta 27 can pass from the source of radiation 26 to the region to be depicted of the patient 13 and after interaction with the region to be depicted strike the detector 28. In this way acquisition data of the region to be depicted can be acquired in the form of projection data via the acquisition unit.

The controller 30 is designed for receiving the acquisition data acquired from the acquisition unit. The controller 30 is designed for controlling the medical imaging device 1. The controller 30 has the data processing unit 35, the computer-readable medium 32 and the processor system 36. The controller 30, in particular the data processing unit 35, is formed by a data processing system, which has a computer.

The controller 30 has the image reconstruction device 34. A medical image data set can be reconstructed via the image reconstruction device 34 based on the acquisition data. The medical imaging device 1 has an input device 38 and an output device 39, which are each connected to the controller 30. The input device 38 is designed for inputting control information, for example image reconstruction parameters, examination parameters or the like. The output device 39 is designed in particular for outputting control information, images and/or acoustic signals.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for selecting a protocol for a medical imaging examination, the medical imaging examination being a medical computerized tomography imaging examination, the method comprising:
providing a plurality of protocols;
providing a classification system for medical imaging examinations, the medical imaging examinations being medical computerized tomography imaging examinations, including a plurality of hierarchically ordered categories,
each respective category of the plurality of hierarchically ordered categories including at least one node, at least one of
assigned to a node of a next relatively higher category, and
including at least one node of a next relatively lower category assigned to the respective category,
the medical imaging examination being identifyable by a set of nodes, including at most one node from each respective category of the plurality of hierarchically ordered categories, and the classification system including a plurality of nodes, to which one respective protocol of the plurality of protocols is assigned;
determining a node, from a quantity of the plurality of nodes, belonging to a set of nodes by which the medical imaging examination is identifyable, and to which one respective protocol is assigned whose category relative to respective categories of other respective nodes of the quantity is relatively lowest; and
selecting the protocol, assigned to the determined node, for the medical imaging examination.

2. The method of claim 1, wherein the classification system includes, as the plurality of hierarchically ordered categories, at least one of at least three categories and exactly three categories.

3. The method of claim 1, wherein the classification system includes three or more categories chosen from the plurality of hierarchically ordered categories, including a first category, relating to a region of a body of a patient to be examined, a second category relating to an anatomical focus of the medical imaging examination, and a third category relating to an issue of the medical imaging examination.

4. The method of claim 1, further comprising:
providing an examination request relating to the medical imaging examination; and
determining the set of nodes by which the medical imaging examination is identifyable, based on the examination request.

5. The method of claim 1, further comprising:
providing a set of training data records, each training data record of the set of training data records including an examination request for medical imaging; and
determining the classification system based on the set of training data records and a machine learning algorithm.

6. The method of claim 5, wherein each respective training data record of the set of training data records includes a respective protocol assigned to the examination request; and wherein the protocols of the plurality of protocols are assigned to the respective nodes of the plurality of nodes based on the set of training data records and a machine learning algorithm.

7. The method of claim 5, wherein the set of training data records includes at least one of examination requests and protocols of at least two different medical imaging devices, to carry out the medical imaging examination.

8. A data processing unit for selecting a protocol for a medical imaging examination, the medical imaging examination being a medical computerized tomography imaging examination, comprising:
a protocol providing unit to provide a plurality of protocols;
a classification system providing unit to provide a classification system for medical imaging examinations, the medical imaging examinations being medical computerized tomography imaging examinations, including a plurality of hierarchically ordered categories, each respective category of the plurality of hierarchically ordered categories including at least one node, at least one of assigned to a node of a next relatively higher category and including at least one node of a next relatively lower category assigned to the respective category,
the medical imaging examination being identifyable by a set of nodes, including at most one node from each respective category of the plurality of categories,
the classification system including a plurality of nodes, to which one respective protocol of the plurality of protocols is assigned,
a node determining unit to determine a node from a quantity of the plurality of nodes, belonging to a set of nodes by which the medical imaging examination is identifyable, and to which one protocol respectively is assigned whose respective category respective categories of other respective nodes of the quantity is relatively lowest; and
a protocol selecting unit to select the protocol, assigned to the determined node, for the medical imaging examination.

9. The data processing unit of claim 8, further comprising:
an examination request-providing unit to provide an examination request, relating to the medical imaging examination; and
a node set determining unit to determine the set of nodes by which the medical imaging examination is identifyable, based on the examination request.

10. The data processing unit of claim 8, further:
a training data record providing unit to provide a set of training data records, each respective training data record of the set of training data records including an examination request for medical imaging; and
a classification system determining unit to determine the classification system based on the set of training data records and a machine learning algorithm.

11. A medical imaging device, including at least one processor as the data processing unit of claim 8.

12. A medical imaging device, including at least one processor as the data processing unit of claim 9.

13. The medical imaging device of claim 11, selected from an imaging modalities group consisting of an X-ray device, a C-arm X-ray device, a computerized tomography device, a molecular imaging device, a single photon emission computerized tomography device, a positron emission tomography device, a magnetic resonance tomography device and a combination of at least one of an X-ray device, a C-arm X-ray device, a computerized tomography device, a molecular imaging device, a single photon emission computerized tomography device, a positron emission tomography device, and a magnetic resonance tomography device.

14. A non-transitory storage device of a data processing system, including a computer program including program segments to carry out the method of claim 1 when the computer program is run by the data processing system.

15. A non-transitory computer-readable medium, storing program segments, readable and runnable by a data processing system, to carry out the method of claim 1 when the program segments are run by the data processing system.

16. The method of claim 4, further comprising:
providing a set of training data records, each training data record of the set of training data records including an examination request for medical imaging; and
determining the classification system based on the set of training data records and a machine learning algorithm.

17. The method of claim 16, wherein each respective training data record of the set of training data records includes a respective protocol assigned to the examination request; and wherein the protocols of the plurality of protocols are assigned to the respective nodes of the plurality of nodes based on the set of training data records and a machine learning algorithm.

18. The method of claim 6, wherein the set of training data records includes at least one of examination requests and protocols of at least two different medical imaging devices, to carry out the medical imaging examination.

19. The data processing unit of claim 9, further:
a training data record providing unit to provide a set of training data records, each respective training data record of the set of training data records including an examination request for medical imaging; and
a classification system determining unit to determine the classification system based on the set of training data records and a machine learning algorithm.

20. A medical imaging device, including at least one processor as the data processing unit of claim 10.

21. The medical imaging device of claim 12, selected from an imaging modalities group consisting of an X-ray device, a C-arm X-ray device, a computerized tomography device, a molecular imaging device, a single photon emission computerized tomography device, a positron emission tomography device, a magnetic resonance tomography device and a combination of at least one of an X-ray device, a C-arm X-ray device, a computerized tomography device, a molecular imaging device, a single photon emission computerized tomography device, a positron emission tomography device, and a magnetic resonance tomography device.

22. A non-transitory storage device of a data processing system, including a computer program including program segments to carry out the method of claim 4 when the computer program is run by the data processing system.

23. A non-transitory computer-readable medium, storing program segments, readable and runnable by a data processing system, to carry out the method of claim 4 when the program segments are run by the data processing system.
